(19) United States
(12) Patent Application Publication
(10) Pub. No.: US 2015/0295000 A1
(43) Pub. Date: Oct. 15, 2015

(54) IMAGING APPARATUS

(71) Applicant: FUJIFILM CORPORATION, TOKYO (JP)

(72) Inventors: SHUICHI ISHII, KANAGAWA (JP); TAKASHI YANO, KANAGAWA (JP); ISSEI SUZUKI, KANAGAWA (JP); YOSHIAKI ISHIMARU, KANAGAWA (JP); YASUNORI OHTA, KANAGAWA (JP)

(21) Appl. No.: 14/662,253

(22) Filed: Mar. 19, 2015

(30) Foreign Application Priority Data
Apr. 9, 2014 (JP) 2014-080018

(51) Int. Cl. H01L 27/146; A61B 5/00

(52) U.S. Cl. CPC H01L 27/14618; A61B 5/0084

(57) ABSTRACT

There is provided an imaging apparatus in which improvements in the moisture-proof and insulation properties of an imaging device and miniaturization of the entire apparatus can be realized. The imaging apparatus is configured to include: an imaging device in which a plurality of photoelectric conversion elements are arrayed; a substrate on which the imaging device is provided and which has a larger outer shape than the imaging device; a transparent cover member that is provided on an opposite surface side to a surface of the imaging device facing the substrate and has a larger outer shape than the imaging device; and a sealing resin that fills a gap between the substrate and the cover member in order to seal a side surface of the imaging device.

### CROSS-REFERENCE TO RELATED APPLICATIONS

The present application claims priority under 35 U.S.C. §119 to Japanese Patent Application No. 2014-080018, filed on Apr. 9, 2014. The above application is hereby expressly incorporated by reference, in its entirety, into the present application.

### TECHNICAL FIELD

The present disclosure relates to an imaging apparatus including an imaging device and a cover member.

### DESCRIPTION OF THE BACKGROUND ART

Medical diagnosis using an electronic endoscope has been performed in the medical field. For example, an insertion unit that is inserted into the body of a patient is provided in an electronic endoscope called a scope. An imaging apparatus configured to include an imaging optical system, an imaging device for capturing an optical image formed by the imaging optical system, and the like is provided in the internal space of a tip portion of the insertion unit. Since the electronic endoscope used for medical diagnosis is subjected to a cleaning and disinfecting process in a cleaning apparatus or a sterilization process under a high-temperature and high-pressure environment in an autoclave, the imaging apparatus in the tip portion of the insertion unit, in particular, the imaging device is generally made to have a moisture-proof (waterproof) and insulating structure.

For example, JP2008-219854A discloses an imaging apparatus including an imaging device, a cover glass that is bonded to the imaging surface (main surface) side of the imaging device by a transparent adhesive and covers a light receiving region provided on the imaging surface, and a sealing resin that covers a side surface of the cover glass and a region of the imaging surface of the imaging device that is not covered by the cover glass. It is possible to improve the moisture-proof and insulation properties of the imaging device using the sealing resin. JP1996-181298A (JP-H08-181298A) discloses an imaging apparatus including a substrate, an imaging device bonded to the top surface of the substrate, a bonding wire for electrically connecting the substrate and the imaging device to each other, a transparent plastic that covers the image area of the imaging device, a cover glass that is provided on the transparent plastic and is larger than the outer shape of the substrate, and a sealing resin that covers a connecting member on the back surface side of the cover glass. In the imaging apparatus disclosed in JP1996-181298A (JP-H08-181298A), the side surfaces of the substrate, the imaging device, and the transparent plastic are covered by the sealing resin. Therefore, it is possible to improve the moisture-proof and insulation properties of the imaging device.

### SUMMARY

Incidentally, miniaturization, such as through wiring or package-less mounting, of the imaging apparatus provided in the electronic endoscope is noticeable. However, even if the size of the imaging apparatus is reduced, the quality, such as moisture resistance, cannot be sacrificed. The imaging apparatus disclosed in JP2008-219854A is effective when the outer shape of the cover glass is smaller than the imaging device, but there is a problem that the side surface of the imaging device cannot be sealed with the sealing resin.

The imaging apparatus disclosed in JP1996-181298A (JP-H08-181298A) is effective when the outer shape of the cover glass is larger than the imaging device, and the side surface of the imaging device can be sealed with the sealing resin. However, it is necessary to provide a transparent plastic between the imaging device and the cover glass. Therefore, there is a problem that it is difficult to reduce the size of the imaging apparatus disclosed in JP1996-181298A (JP-H08-181298A) due to an increase in thickness caused by providing the cover glass and the transparent plastic, that is, caused by forming a transparent member in a double structure. In addition, in the imaging apparatus disclosed in JP1996-181298A (JP-H08-181298A), not only the side surfaces of the imaging device and the like but also the bonding wire is covered by the sealing resin. Therefore, there is a problem that the size of the imaging apparatus disclosed in JP1996-181298A (JP-H08-181298A) is increased by the amount by which the connecting member is covered by the sealing resin.

The disclosure has been made in view of such a situation, and it is to provide an imaging apparatus in which improvements in the moisture-proof and insulation properties of an imaging device and miniaturization of the entire apparatus can be realized.

In order to achieve the disclosure, there is provided an imaging apparatus including: an imaging device in which a plurality of photoelectric conversion elements are arrayed; a substrate on which the imaging device is provided and which has a larger outer shape than the imaging device; a transparent cover member that is provided on an opposite surface side to a surface of the imaging device facing the substrate and has a larger outer shape than the imaging device; and a sealing resin that fills a gap between the substrate and the cover member in order to seal a side surface of the imaging device.

According to the imaging apparatus of the disclosure, the outer shapes of the cover member and the substrate are formed so as to be larger than the outer shape of the imaging device, and the side surface of the imaging device is sealed by filling a gap between the cover member and the substrate with the sealing resin. Therefore, it is possible to improve the moisture-proof and insulation properties of the imaging device. In addition, since it is not necessary to provide two kinds of transparent members (cover glass and transparent plastic) unlike in the imaging apparatus disclosed in JP1996-181298A (JP-H08-181298A), the imaging apparatus can be made to have a smaller thickness than in the related art. That is, the imaging apparatus can be miniaturized.

In the imaging apparatus according to the aspect of the disclosure, preferably, the sealing resin is provided on an inner side rather than outer peripheries of the substrate and the cover member when sizes of the outer shapes of the substrate and the cover member are the same. In this case, it is possible to seal the side surface of the imaging device with the sealing resin.

In the imaging apparatus according to the aspect of the disclosure, preferably, when sizes of the outer shapes of the substrate and the cover member are different, the sealing resin is provided on an inner side rather than an outer periphery of one of the substrate and the cover member having the larger outer shape. In this case, it is possible to seal the side surface of the imaging device with the sealing resin.

In the imaging apparatus according to the aspect of the disclosure, preferably, a pattern that has a frame shape surrounding the sealing resin and regulates a flow of the sealing resin toward the outer periphery of one of the substrate and the cover member having the larger outer shape is formed on a surface, which faces the imaging device, of one of the substrate and the cover member having the larger outer shape. In this case, the occurrence of a situation is prevented in which the sealing resin filling a gap between the cover member and the substrate flows out from between the cover member and the substrate before the sealing resin is solidified.

In the imaging apparatus according to the aspect of the disclosure, it is preferable that the pattern is formed by plating or deposition when the cover member has a larger outer shape. In this case, it is possible to form a pattern on the cover member.

In the imaging apparatus according to the aspect of the disclosure, it is preferable that the sealing resin contains a filler having higher thermal conductivity than the sealing resin. In this case, since the heat dissipation performance of the sealing resin can be improved, heat generated from the imaging device is dissipated to the outside of the imaging device through the sealing resin.

In the imaging apparatus according to the aspect of the disclosure, it is preferable that a tensile modulus of elasticity of the sealing resin is 100 Mpa or less. In this case, even if the volume of a void or the like contained in the sealing resin is increased or decreased due to pressurization or decompression, it is possible to absorb the increase or decrease in the volume of the void or the like with the sealing resin. As a result, damage caused to the imaging device due to the increase or decrease in the volume of the void or the like is prevented. Here, the specification when measuring the tensile modulus of elasticity is “ASTMD 638”.

In the imaging apparatus according to the aspect of the disclosure, preferably, a through wiring portion is formed in the imaging device, a connecting portion electrically connected to the through wiring portion is formed on a surface of the substrate facing the imaging device, and the imaging device and the substrate are electrically connected to each other through the through wiring portion and the connecting portion. In this case, since it is not necessary to cover a bonding wire with a sealing resin unlike in the imaging apparatus disclosed in JP1996-181298A (JP-H08-181298A), it is possible to further miniaturize the imaging apparatus.

In the imaging apparatus according to the aspect of the disclosure, it is preferable that the imaging device is of a Back Side Illumination type. In this case, compared with a general top surface irradiation type imaging device, it is possible to obtain a bright image even with the same amount of light.

In the imaging apparatus according to the aspect of the disclosure, it is preferable that a spacer is provided between the imaging device and the cover member. Also in the imaging apparatus in which the spacer is provided, it is possible to seal the side surface of the imaging device with the sealing resin.

In the imaging apparatus according to the aspect of the disclosure, it is preferable that the cover member is formed of glass or transparent resin.

Through the imaging apparatus of the disclosure, it is possible to realize improvements in the moisture-proof and insulation properties of the imaging device and miniaturization of the entire apparatus.

### BRIEF DESCRIPTION OF THE DRAWINGS

FIG. 1 is a perspective view of an endoscope apparatus.

FIG. 2 is a front view of an insertion unit tip portion of the electronic endoscope.

FIG. 3 is a cross-sectional view of a flexible tube portion of the electronic endoscope.

FIG. 4 is a cross-sectional view of the insertion unit tip portion of the electronic endoscope.

FIGS. 5A and 5B are enlarged views of an imaging apparatus of a first embodiment.

FIG. 6 is a cross-sectional view taken along line A-A of FIG. 5B.

FIG. 7 is an enlarged view of a cross-section of a pixel of an imaging device.

FIG. 8 is an enlarged view of a portion surrounded by a dotted circle in the imaging apparatus shown in FIG. 6.

FIG. 9 is a top view of an imaging apparatus of a second embodiment.

FIG. 10 is a cross-sectional view of the imaging apparatus of the second embodiment.

FIG. 11 is a cross-sectional view of an imaging apparatus of a third embodiment.

FIG. 12 is an enlarged view of a portion surrounded by a dotted circle in the imaging apparatus shown in FIG. 11.

FIG. 13 is a cross-sectional view of an imaging apparatus that is a modification example of the imaging apparatus of the third embodiment.

FIG. 14 is a cross-sectional view of an imaging apparatus of a fourth embodiment.

FIG. 15 is a cross-sectional view of the main part of the imaging apparatus of the fourth embodiment.

FIG. 16 is an enlarged view of a portion surrounded by a dotted circle in the imaging apparatus shown in FIG. 15.

FIG. 17 is a cross-sectional view of an imaging apparatus that is a modification example of the imaging apparatus of the fourth embodiment.

FIG. 18 is an enlarged view of a portion surrounded by a dotted circle of the imaging apparatus shown in FIG. 17.

FIG. 19 is a schematic diagram of a capsule system including the imaging apparatus of the disclosure.

FIG. 20 is a schematic diagram of a capsule system of another embodiment.

### DESCRIPTION OF THE EXEMPLARY EMBODIMENTS

Hereinafter, an imaging apparatus according to the disclosure will be described with reference to the accompanying diagrams.

[Overall Configuration of an Endoscope Apparatus]

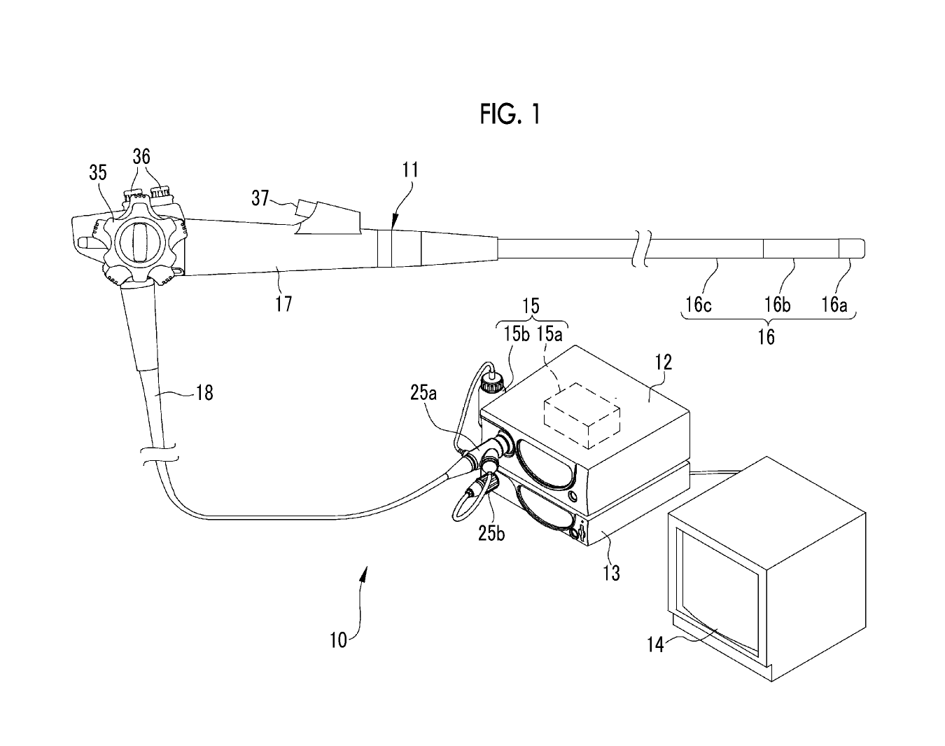

FIG. 1 is a perspective view showing the appearance of an endoscope apparatus (also referred to as an endoscope system) 10. The endoscope apparatus 10 includes an electronic endoscope 11 as a scope (here, a soft endoscope) for imaging an observation part inside the body of a patient, a light source device 12, a processor device 13, a monitor 14, and an air and water supply device 15.

The light source device 12 supplies illumination light for illuminating an observation part to the electronic endoscope 11. The processor device 13 generates image data of the observation part based on an imaging signal obtained by the electronic endoscope 11, and outputs the image data to the monitor 14. The monitor 14 displays an observation image of the observation part based on the image data input from the processor device 13. The air and water supply device 15 includes an air supply pump 15a, which is provided in the light source device 12 and generates pressure for sending fluids, such as air and cleaning water, and a cleaning water tank 15b, which is provided outside the light source device 12 and stores cleaning water.

The electronic endoscope 11 includes a flexible insertion unit 16 that is inserted into the body of a patient, an operating unit 17 that is provided continuously to a base portion of the insertion unit 16 and is used in order to grip the electronic endoscope 11 and operate the insertion unit 16, and a universal cord 18 for connecting the operating unit 17 to the light source device 12 and the processor device 13.

As will be described in detail later, an optical system, an imaging device, and the like that are used for illumination and imaging of the observation part are provided in an insertion unit tip portion 16a that is a tip portion of the insertion unit 16. A bending portion 16b that can bend is provided continuously to the rear end of the insertion unit tip portion 16a. In addition, a flexible tube portion 16c having flexibility is provided continuously to the rear end of the bending portion 16b.

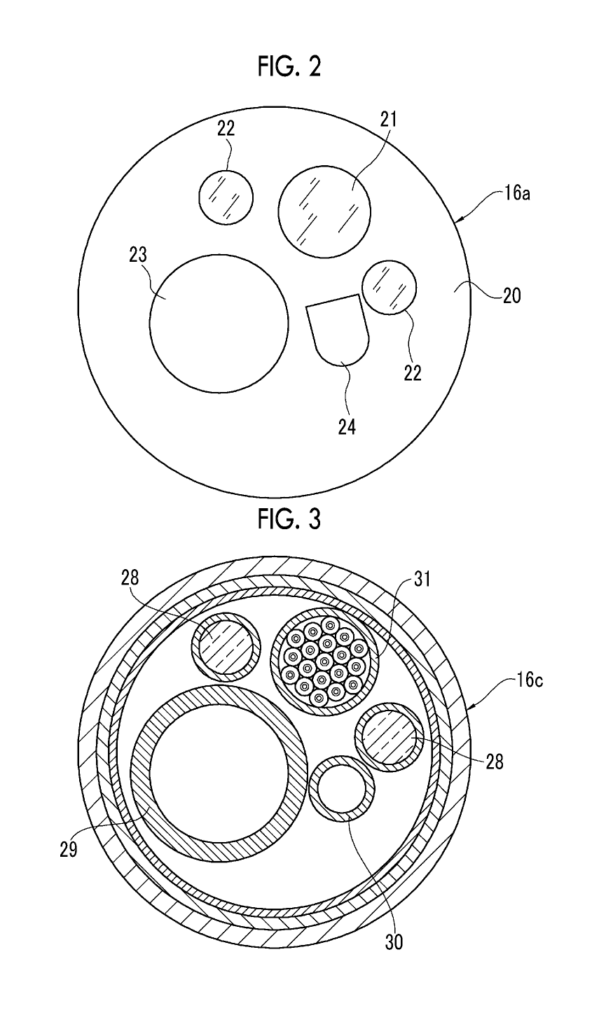

FIG. 2 is a front view of the insertion unit tip portion 16a. As shown in FIG. 2, an observation window 21, an illumination window 22, a forceps exit 23, and a spraying nozzle 24 are provided in a tip cover 20 of the insertion unit tip portion 16a. The optical system, the imaging device, and the like described above are provided behind the observation window 21. Two illumination windows 22 are disposed at two symmetrical positions with respect to the observation window 21, so that illumination light supplied from the light source device 12 is emitted to the observation part inside the body of the patient. The forceps exit 23 communicates with a forceps entrance 37 (refer to FIG. 1) of the operating unit 17. Air and cleaning water supplied from the air and water supply device 15 are sprayed toward the observation window 21 through the spraying nozzle 24, thereby removing the dirt adhering to the observation window 21.

FIG. 3 is a cross-sectional view of the flexible tube portion 16c. As shown in FIG. 3, a plurality of built-in products, such as a light guide 28, a forceps channel 29, an air and water supply channel 30, and a cable 31 are loosely inserted into the flexible tube portion 16c. The light guide 28 serves to guide light from the light source device 12 to the illumination window 22. The forceps channel 29 communicates with the forceps exit 23 and the forceps entrance 37. The air and water supply channel 30 sends the air and cleaning water, which are supplied from the air and water supply device 15, to the spraying nozzle 24. The cable 31 electrically connects the processor device 13 and the imaging device of the electronic endoscope 11 to each other.

Referring back to FIG. 1, an angle knob 35, an operation button 36, the forceps entrance 37, and the like are provided in the operating unit 17. The angle knob 35 is rotated when adjusting the bending direction and bending amount of the bending portion 16b. The operation button 36 is used for various operations, such as air supply, water supply, and suction. The forceps entrance 37 communicates with the forceps channel 29.

The light guide 28, the air and water supply channel 30, the cable 31, and the like described above are provided in the universal cord 18. A connector portion 25a connected to the light source device 12 and the air and water supply device 15 and a connector portion 25b connected to the processor device 13 are provided in a tip portion of the universal cord 18. Therefore, through the connector portion 25a, illumination light is supplied from the light source device 12 to the light guide 28, and air and water are supplied from the air and water supply device 15 to the air and water supply channel 30. In addition, an imaging signal obtained by the electronic endoscope 11 is input to the processor device 13 through the connector portion 25b.

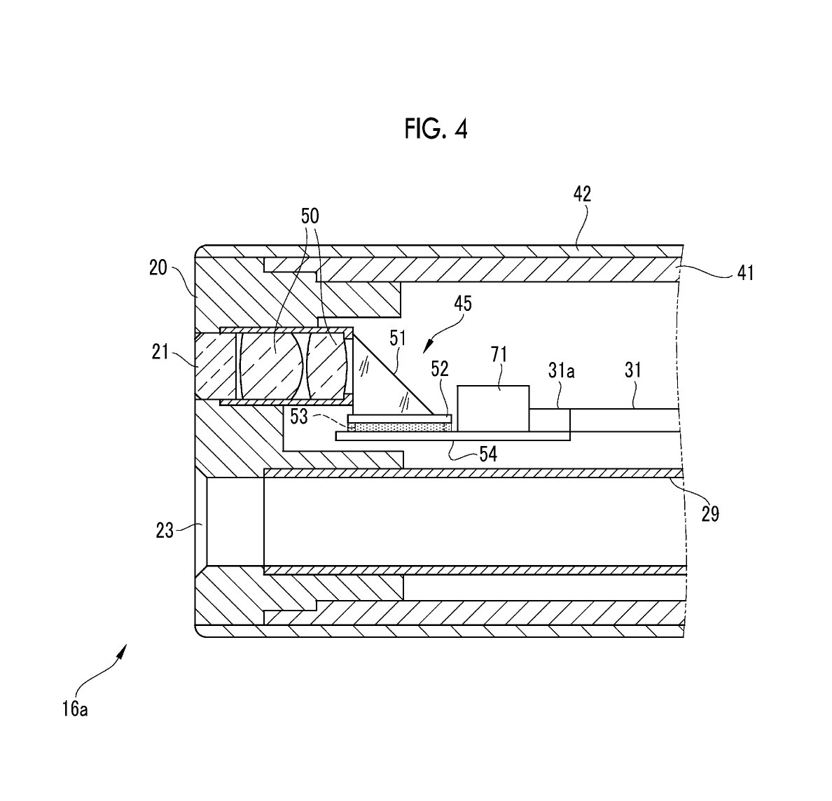

FIG. 4 is a cross-sectional view of the insertion unit tip portion 16a. As shown in FIG. 4, the insertion unit tip portion 16a is configured to include a cylindrical body 41, the tip cover 20 that closes an opening on the tip side of the cylindrical body 41, a rubber 42 that covers the outer periphery of the cylindrical body 41 and the tip cover 20, and various built-in products that are provided in the internal space of the insertion unit tip portion 16a.

In the internal space of the insertion unit tip portion 16a, the light guide 28 (not shown in FIG. 4), the forceps channel 29, the air and water supply channel 30 (not shown in FIG. 4), the cable 31, and the like are inserted, and an imaging apparatus 45 is provided. In addition, although not shown in the diagram, the internal space of the insertion unit tip portion 16a is filled with resin, so that various built-in products in the internal space are fixed by the resin.

The forceps channel 29 is connected to the forceps exit 23 of the tip cover 20. Although not shown in FIG. 4, an illumination lens is provided behind the illumination window 22, and an emitting end of the light guide 28 faces the illumination lens. Although not shown in FIG. 4, the air and water supply channel 30 is connected to the spraying nozzle 24. One end of the light guide 28, one end of the forceps channel 29, and one end of the air and water supply channel 30 are fixed to the tip cover 20, and the other ends of the light guide 28, the forceps channel 29, and the air and water supply channel 30 are connected to the light source device 12, the forceps entrance 37, and the air and water supply device 15, respectively, through the insides of the bending portion 16b, the flexible tube portion 16c, the operating unit 17, and the like.

[Configuration of an Imaging Apparatus of a First Embodiment]

The imaging apparatus 45 is disposed behind the observation window 21. The imaging apparatus 45 mainly includes an observation optical system (also referred to as an objective optical system) 50, a prism 51, a cover glass 52, an imaging device 53, and a back substrate 54. Although the cover glass 52, the imaging device 53, and the back substrate 54 are formed in a rectangular shape in the present embodiment, the shape is not particularly limited.

The observation optical system 50 is housed in a lens barrel fixed to the observation window 21 of the tip cover 20. The observation optical system 50 makes image light of the observation part, which is incident from the observation window 21, incident on the prism 51.

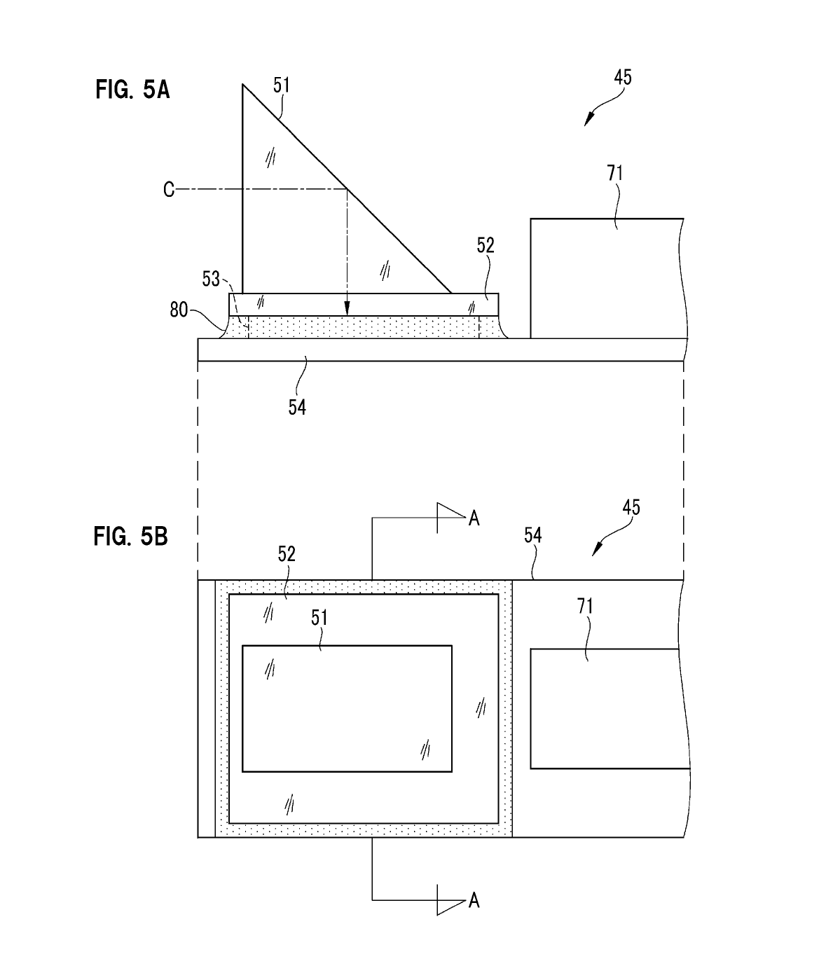

FIGS. 5A and 5B are enlarged views of the imaging apparatus 45. As shown in FIG. 5A and FIG. 5B, the prism 51 makes image light C incident from the observation optical system 50 bend by 90° and be emitted toward the imaging device 53. As a result, the image light C (optical image) of the observation part is formed on the imaging device 53 through the cover glass 52. The incidence surface of the image light C of the prism 51 is bonded and fixed to the lens barrel of the observation optical system 50.

The cover glass 52 is equivalent to one form of a transparent cover member of the disclosure. One surface of the cover glass 52 is bonded and fixed to the imaging surface of the imaging device 53, and the other surface of the cover glass 52 is bonded and fixed to the emission surface of the image light C of the prism 51. Accordingly, the imaging surface of the imaging device 53 is protected by the cover glass 52, and the imaging device 53 is fixed to the prism 51 through the cover glass 52. The cover glass 52 is formed so as to have a larger outer shape than the imaging device 53. Specifically, the cover glass 52 is formed such that the position of the outer periphery of the cover glass 52 is located outside the position of the outer periphery of the imaging device 53. Instead of the cover glass 52, it is possible to use a cover member formed of a transparent resin.

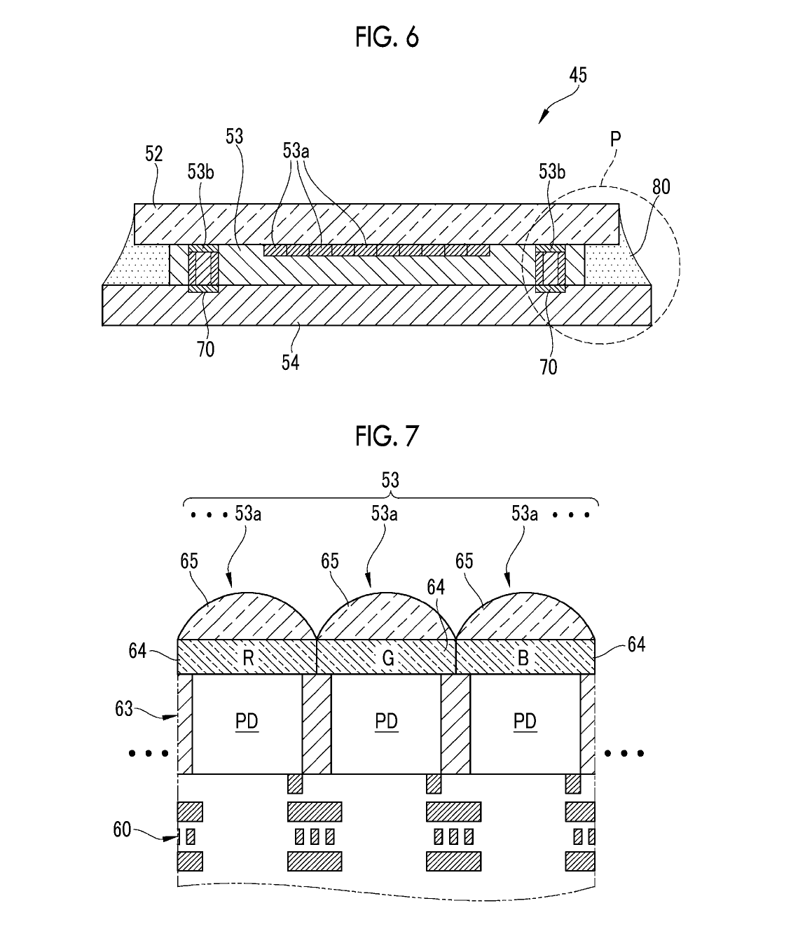

FIG. 6 is a cross-sectional view taken along the line A-A of FIG. 5B. In FIG. 6, the prism 51 is not shown (the same for other cross-sectional views to be described below). As shown in FIG. 6, a plurality of pixels 53a formed by a plurality of photoelectric conversion elements (photodiodes) PD arrayed in a two-dimensional manner are formed on the imaging surface of the imaging device 53 (refer to FIG. 7). The imaging device 53 converts the image light C incident through the cover glass 52 into an electrical imaging signal in each pixel 53a, and outputs the electrical imaging signal.

FIG. 7 is an enlarged view of a cross-section of the pixel 53a of the imaging device 53. The imaging device 53 of the present embodiment is a Back Side Illumination type complementary metal oxide semiconductor (CMOS) type imaging device, and has a structure in which a light receiving layer 63, in which the photoelectric conversion elements PD are arrayed for the respective pixels 53a, is formed on a wiring layer 60. A microlens 65 and a color filter 64 of each color of red (R), green (G), and blue (B) are formed on each photoelectric conversion element PD. In the Back Side Illumination type imaging device 53, the image light C can be incident from the back surface side on which there is no wiring layer 60 in the light receiving layer 63. Therefore, compared with a general top surface irradiation type CMOS type imaging device, it is possible to obtain a bright image even with the same amount of light.

The imaging device 53 is not limited to the Back Side Illumination type CMOS imaging device, and may be a top surface irradiation type CMOS type imaging device or may be a charge coupled device (CCD) type imaging device.

Referring back to FIG. 6, in the imaging device 53, a through wiring portion 53b for electrical connection with the back substrate 54 is formed in a region other than the formation region of the pixel 53a. The through wiring portion 53b is electrically connected to the wiring layer 60 or the like through a wiring line (not shown).

The back substrate 54 is equivalent to one form of a substrate of the disclosure, and is fixed to the back surface of the imaging device 53 on a side opposite to the imaging surface. In other words, the imaging device 53 is fixed onto the back substrate 54. The back substrate 54 is formed so as to have a larger outer shape than the imaging device 53 and the cover glass 52. Specifically, the back substrate 54 is formed such that the position of the outer periphery of the back substrate 54 is located on the outer side rather than the position of the outer periphery of the imaging device 53 and the position of the outer periphery of the cover glass 52. The back substrate 54 may be either a rigid substrate or a flexible printed circuit (FPC) substrate.

On the back substrate 54, a connecting portion 70, such as a bump or a land, is formed, and a peripheral circuit 71 (refer to FIG. 4 and FIGS. 5A and 5B) is fixed or electrically connected in addition to the imaging device 53 described above.

The connecting portion 70 is electrically connected to the through wiring portion 53b of the imaging device 53. Therefore, the imaging device 53 and the back substrate 54 are electrically connected to each other through the through wiring portion 53b and the connecting portion 70.

The peripheral circuit 71 controls the driving of the imaging device 53. As shown in FIG. 4, a cable tip portion 31a that is a tip portion of the cable 31 is electrically connected to the peripheral circuit 71. Therefore, an imaging signal output from the imaging device 53 is output to the cable 31 through the peripheral circuit 71. In addition, when the imaging device 53 is a CCD type imaging device, the imaging signal output from the imaging device 53 is output to the cable 31 after conversion into the imaging signal or buffering in the peripheral circuit 71.

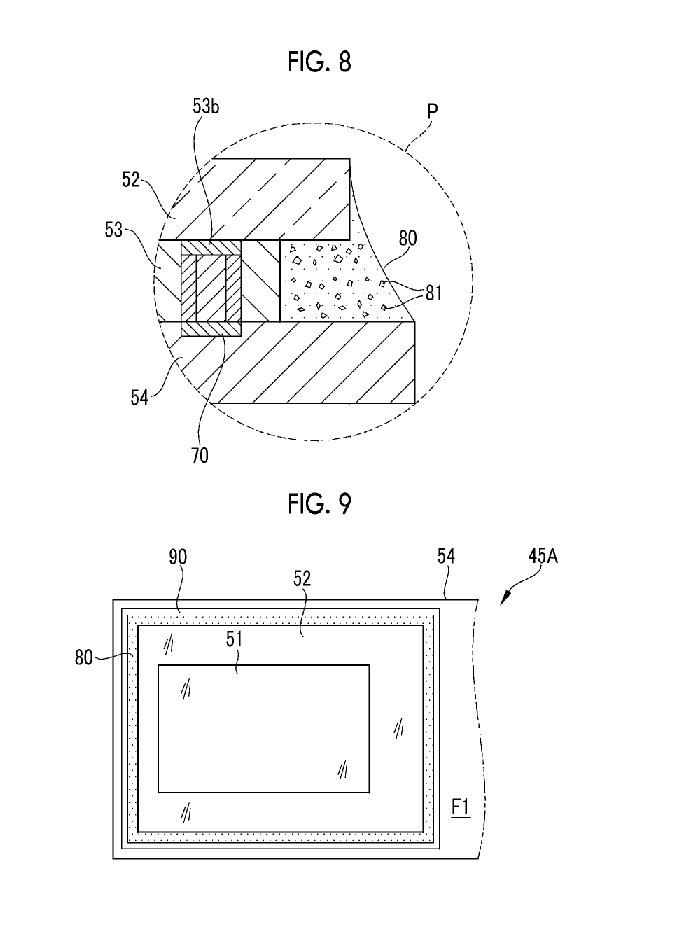

FIG. 8 is an enlarged view of a portion surrounded by a dotted circle P in the imaging apparatus 45 shown in FIG. 6. As shown in FIGS. 6 and 8, the cover glass 52 and the back substrate 54 are formed so as to have larger outer shapes than the imaging device 53. For this reason, a gap (space) is formed between the cover glass 52 and the back substrate 54 by the cover glass 52, the back substrate 54, and the side surface of the imaging device 53, and a sealing resin 80 having moisture-proof (waterproof) and insulation properties fills the gap. Specifically, the sealing resin 80 is solidified after filling the gap with the sealing resin 80 using a capillary phenomenon based on surface tension. As a result, the side surface of the imaging device 53 is sealed with the sealing resin 80.

In this case, in the imaging apparatus 45, since the back substrate 54 is formed so as to have a larger outer shape than the cover glass 52, the sealing resin 80 is provided on the inner side rather than the outer periphery of the back substrate 54. In addition, although the position of the outer periphery of the back substrate 54 is located on the outer side rather than the position of the outer periphery of the cover glass 52 in the entire outer periphery of the cover glass 52 and the back substrate 54 in the present embodiment, the position of the outer periphery of the cover glass 52 may be partially the same as the position of the outer periphery of the back substrate 54 (for example, refer to FIG. 15). Also in this case, the sealing resin 80 is provided on the inner side rather than the outer periphery of the back substrate 54.

As the sealing resin 80, for example, a soft resin such as a silicon resin having a tensile modulus of elasticity of 100 Mpa or less (ASTMD 638) is used. The reason is as follows. As a part of the sterilization process performed for the electronic endoscope 11 that has been used for medical diagnosis, for example, pressurization or decompression processing using ethylene oxide gas is performed. In this case, however, if space, such as a void, is included in the sealing resin 80, the volume of the void or the like is increased or decreased due to pressurization or decompression. Therefore, by using the soft resin, such as a silicon resin, as the sealing resin 80, it is possible to absorb an increase or decrease in the volume of the void or the like with the sealing resin 80. As a result, damage caused to the imaging device 53 or the like due to the increase or decrease in the volume of the void or the like is prevented.

In addition, the sealing resin 80 contains a filler 81. As the filler 81, alumina, silicon oxide, and the like having higher thermal conductivity than at least the sealing resin 80 are used. Since the heat dissipation performance of the sealing resin 80 can be improved in this manner, heat generated from the imaging device 53 is dissipated to the outside of the imaging device 53 through the sealing resin 80.

[Effect of the Imaging Apparatus of the First Embodiment]

As described above, in the imaging apparatus 45 configured as described above, the outer shapes of the cover glass 52 and the back substrate 54 are formed so as to be larger than the outer shape of the imaging device 53, and the side surface of the imaging device 53 is sealed by filling the gap between the cover glass 52 and the back substrate 54 with the sealing resin 80. Therefore, the imaging device 53 is not exposed to the outside of the imaging apparatus 45. As a result, since the side surface of the imaging device 53 can be made to have a moisture-proof and insulating structure, it is possible to improve the moisture-proof and insulation properties of the imaging device 53. In addition, since it is not necessary to form a transparent member (cover glass and transparent plastic) in a double structure unlike in the imaging apparatus disclosed in JP1996-181298A (JP-H08-181298A), the imaging apparatus 45 can be made to have a smaller thickness than the imaging apparatus disclosed in JP1996-181298A (JP-H08-181298A). That is, the imaging apparatus 45 can be miniaturized. As a result, in the imaging apparatus 45, it is possible to realize both improvements in the moisture-proof and insulation properties of the imaging device 53 and miniaturization.

In the imaging apparatus 45, the imaging device 53 and the back substrate 54 are electrically connected to each other through the through wiring portion 53b and the connecting portion 70. Therefore, it is not necessary to cover a bonding wire with a sealing resin unlike in the imaging apparatus disclosed in JP1996-181298A (JP-H08-181298A) in which the imaging device and the back substrate are electrically connected to each other through a bonding wire. As a result, it is possible to further miniaturize the imaging apparatus 45.

In addition, since a silicon resin or the like having a tensile modulus of elasticity of 100 Mpa or less is used as the sealing resin 80, it is possible to absorb an increase or decrease in the volume of a void or the like in the sealing resin 80, which is caused by pressurization or decompression during sterilization of the electronic endoscope 11, with the sealing resin 80. As a result, damage caused to the imaging device 53 or the like due to the increase or decrease in the volume of the void or the like is prevented.

In addition, since the sealing resin 80 contains the filler 81 having higher thermal conductivity than at least the sealing resin 80, it is possible to improve the heat dissipation performance as well as the moisture resistance of the imaging device 53.

[Imaging Apparatus of a Second Embodiment]

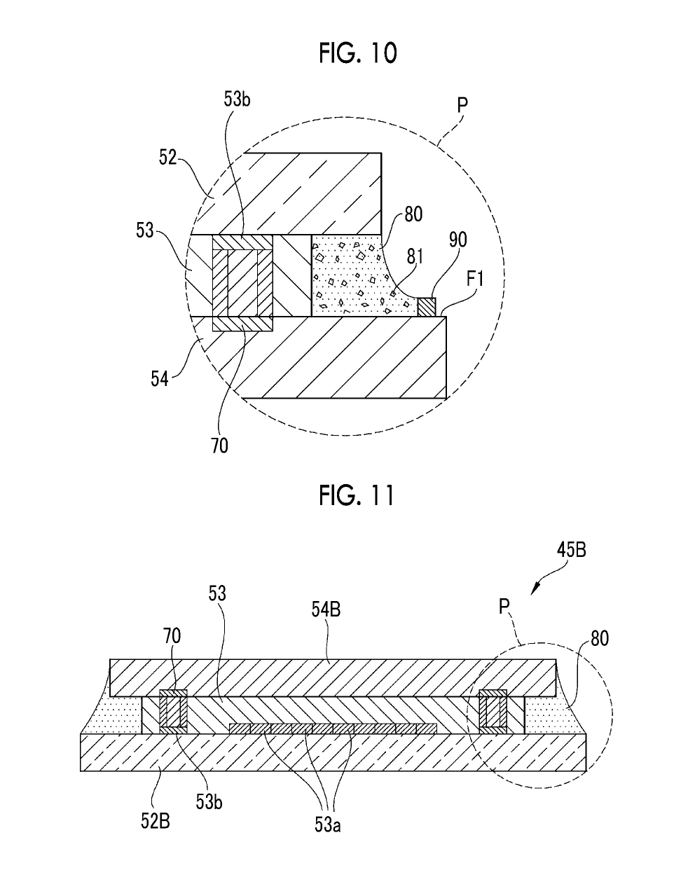

Next, an imaging apparatus 45A of a second embodiment of the disclosure will be described with reference to FIGS. 9 and 10. FIG. 9 is a top view of the imaging apparatus 45A, and FIG. 10 is a cross-sectional view of the imaging apparatus 45A. In the imaging apparatus 45 of the first embodiment described above, the sealing resin 80 fills a gap between the cover glass 52 and the back substrate 54 and is solidified. However, depending on the viscosity of the sealing resin 80 or the like, the sealing resin 80 may flow out from between the cover glass 52 and the back substrate 54 before the used sealing resin 80 is solidified.

Therefore, in the imaging apparatus 45A, a frame-shaped pattern 90 surrounding the sealing resin 80 is formed on an opposite surface F1 of the back substrate 54 facing the imaging device 53. Since the imaging apparatus 45A has basically the same configuration as the imaging apparatus 45 of the first embodiment except that the pattern 90 is formed, components having the same functions and configurations as in the first embodiment described above are denoted by the same reference numerals, and explanation thereof will be omitted.

The pattern 90 is formed, for example, by patterning similar to a wiring pattern (not shown) formed on the back substrate 54, and has a fixed height with respect to the opposite surface F1. The pattern 90 functions as a so-called dam to regulate the flow of the sealing resin 80, which fills a gap between the cover glass 52 and the back substrate 54, toward the outer peripheral side of the back substrate 54. Therefore, the occurrence of a situation is prevented in which the sealing resin 80 filling the gap between the cover glass 52 and the back substrate 54 flows out from between the cover glass 52 and the back substrate 54 before the sealing resin 80 is solidified. As a result, since the side surface of the imaging device 53 is reliably sealed with the sealing resin 80 between the cover glass 52 and the back substrate 54, it is possible to reliably improve the moisture-proof and insulation properties of the imaging device 53.

In addition, since the imaging apparatus 45A has basically the same configuration as the imaging apparatus 45 of the first embodiment except that the pattern 90 is formed, it is possible to obtain the same effects as those described in the first embodiment.

[Imaging Apparatus of a Third Embodiment]

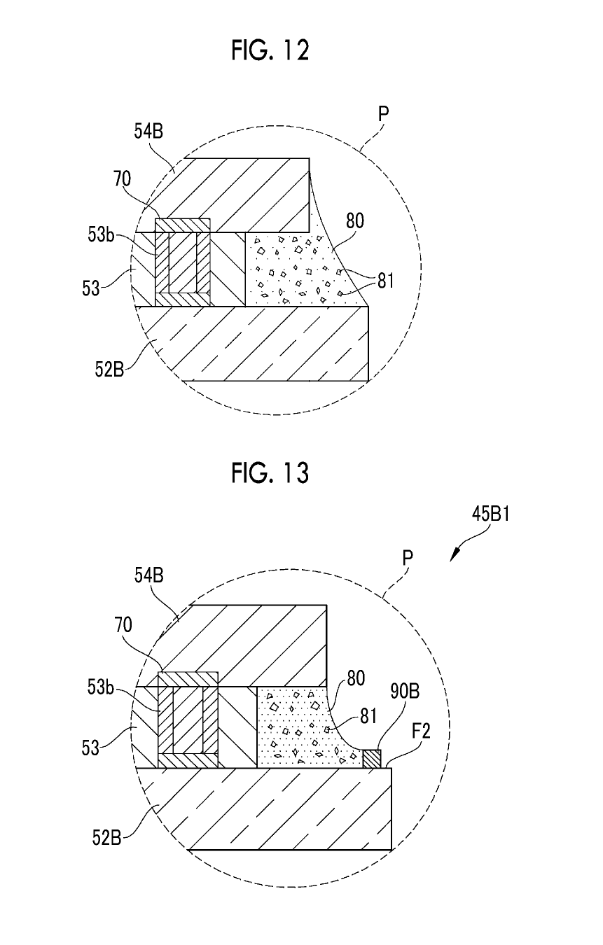

Next, an imaging apparatus 45B of a third embodiment of the disclosure will be described with reference to FIGS. 11 and 12. FIG. 11 is a cross-sectional view of the imaging apparatus 45B, and FIG. 12 is an enlarged view of a portion surrounded by a dotted circle P in the imaging apparatus 45B shown in FIG. 11. In the imaging apparatus 45 of the first embodiment described above, the back substrate 54 is formed so as to have a larger outer shape than the cover glass 52. However, in the imaging apparatus 45B, a cover glass 52B is formed so as to have a larger outer shape than a back substrate 54B. Specifically, the cover glass 52B is formed such that the position of the outer periphery of the cover glass 52B is located on the outer side rather than the position of the outer periphery of the imaging device 53 and the position of the outer periphery of the back substrate 54B.

Since the imaging apparatus 45B has basically the same configuration as the imaging apparatus 45 of the first embodiment except that the cover glass 52B and the back substrate 54B are provided instead of the cover glass 52 and the back substrate 54 in the first embodiment, components having the same functions and configurations as in the first embodiment described above are denoted by the same reference numerals, and explanation thereof will be omitted.

In the imaging apparatus 45B, since the cover glass 52B is formed so as to have a larger outer shape than the back substrate 54B, the sealing resin 80 is provided on the inner side rather than the outer periphery of the cover glass 52B. Therefore, since the side surface of the imaging device 53 is sealed with the sealing resin 80 between the cover glass 52B and the back substrate 54B, it is possible to obtain the same effects as those described in the first embodiment.

In addition, although the position of the outer periphery of the cover glass 52B is located on the outer side rather than the position of the outer periphery of the back substrate 54B in the entire outer periphery of the cover glass 52B and the back substrate 54B in the present embodiment, the position of the outer periphery of the cover glass 52B and the position of the outer periphery of the back substrate 5 may be partially the same.

[Modification Example of the Imaging Apparatus of the Third Embodiment]

FIG. 13 is a cross-sectional view of an imaging apparatus 45B1 that is a modification example of the imaging apparatus 45B of the third embodiment. As shown in FIG. 13, in the imaging apparatus 45B1, a frame-shaped pattern 90B surrounding the sealing resin 80 is formed on an opposite surface F2 of the cover glass 52B facing the imaging device 53. Since the imaging apparatus 45B 1 has basically the same configuration as the imaging apparatus 45B of the third embodiment except that the pattern 90B is formed on the opposite surface F2, components having the same functions and configurations as in the third embodiment described above are denoted by the same reference numerals, and explanation thereof will be omitted.

The pattern 90B has basically the same function as the pattern 90 of the second embodiment, and functions as a so-called dam to regulate the flow of the sealing resin 80, which fills a gap between the cover glass 52B and the back substrate 54B, toward the outer peripheral side of the cover glass 52B. However, since the pattern 90B needs to be formed on the cover glass 52B, the pattern 90B is formed on the opposite surface F2 by plating (for example, black chrome plating) or deposition. Similarly when a cover member formed of a transparent resin instead of the cover glass 52B is used, a pattern is formed by black chrome plating or the like. By forming the pattern 90B on the opposite surface F2, the occurrence of a situation in which the sealing resin 80 filling the gap between the cover glass 52B and the back substrate 54B flows out from between the cover glass 52B and the back substrate 54B before the sealing resin 80 is solidified is prevented as in the second embodiment. As a result, it is possible to obtain the same effects as those described in the second embodiment.

[Imaging Apparatus of a Fourth Embodiment]

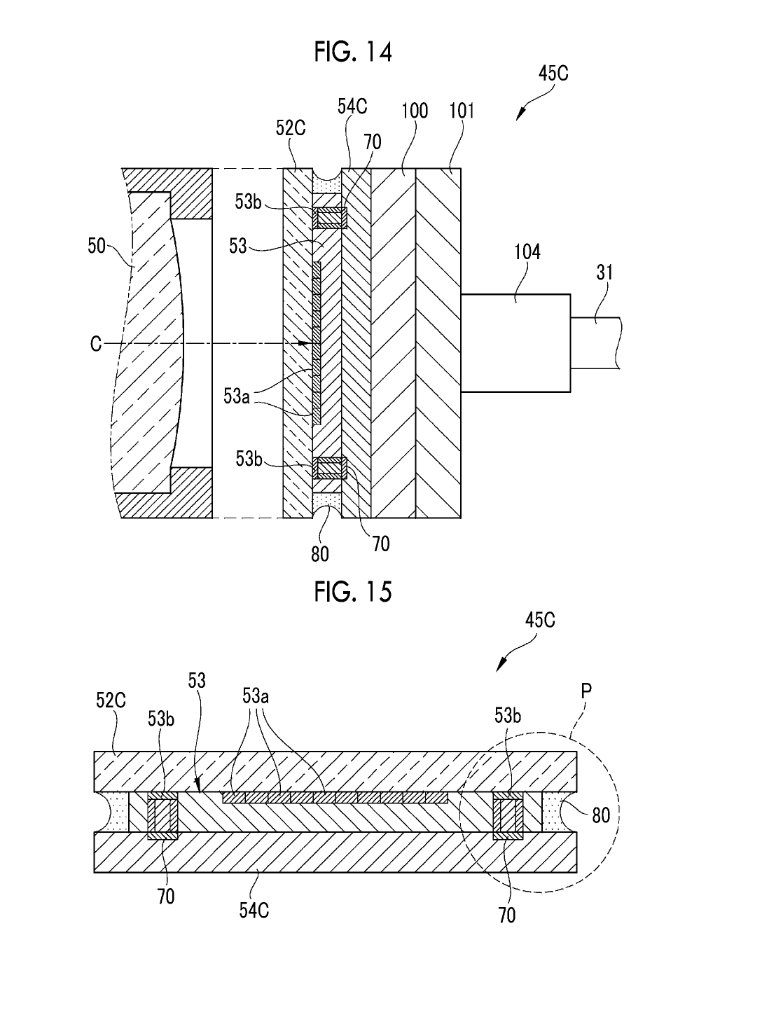

Next, an imaging apparatus 45C of a fourth embodiment of the disclosure will be described with reference to FIG. 14. FIG. 14 is a cross-sectional view of the imaging apparatus 45C. In the imaging apparatus 45 of the first embodiment described above, a so-called transverse structure is adopted in which the imaging device 53 is disposed such that the imaging surface is parallel to the optical axis of the observation optical system 50 and the image light C from the observation optical system 50 is incident on the imaging surface after being bent by 90° by the prism 51. In contrast, in the imaging apparatus 45C, a so-called vertical structure is adopted in which the imaging device 53 is disposed such that the imaging surface is perpendicular to the optical axis of the observation optical system 50. In addition, components having the same functions and configurations as in the first embodiment described above are denoted by the same reference numerals, and explanation thereof will be omitted.

The imaging apparatus 45C mainly includes an observation optical system 50, a cover glass 52C, an imaging device 53, a back substrate 54C, a first peripheral circuit 100, and a second peripheral circuit 101.

One surface of the cover glass 52C is bonded and fixed to the lens barrel of the observation optical system 50, and the other surface of the cover glass 52C is bonded and fixed to the imaging surface of the imaging device 53. Accordingly, the imaging surface of the imaging device 53 is protected by the cover glass 52C, and the imaging device 53 is fixed to the lens barrel of the observation optical system 50 through the cover glass 52C. Similar to each embodiment described above, the cover glass 52C is formed so as to have a larger outer shape than the imaging device 53.

The imaging device 53 is the same as the imaging device 53 of the first embodiment, and includes a plurality of photoelectric conversion elements PD (a plurality of pixels 53a) arrayed in a two-dimensional manner, a through wiring portion 53b, and the like.

The back substrate 54C is equivalent to one form of the substrate of the disclosure, and is fixed to the back surface of the imaging device 53 on the opposite side to the imaging surface. The back substrate 54C is a rigid substrate or an FPC substrate formed in an outer shape that is larger than the imaging device 53 and is the same as the cover glass 52C in size. The back substrate 54C has a connecting portion 70 similar to the back substrate 54 of the first embodiment, and is electrically connected to the imaging device 53 through the connecting portion 70 or the like.

The first peripheral circuit 100 and the second peripheral circuit 101 are fixed to a surface of the back substrate 54C on the opposite side of a surface to which the imaging device 53 is fixed. The first peripheral circuit 100 controls the driving of the imaging device 53. The second peripheral circuit 101 is fixed in a state of being electrically connected to an opposite surface to a surface of the first peripheral circuit 100 facing the back substrate 54C. The second peripheral circuit 101 is connected to the cable 31 through a cable connecting portion 104. Therefore, an imaging signal output from the imaging device 53 is output to the cable 31 through the first and second peripheral circuits 100 and 101 or the like. The functions of the first and second peripheral circuits 100 and 101 are not particularly limited.

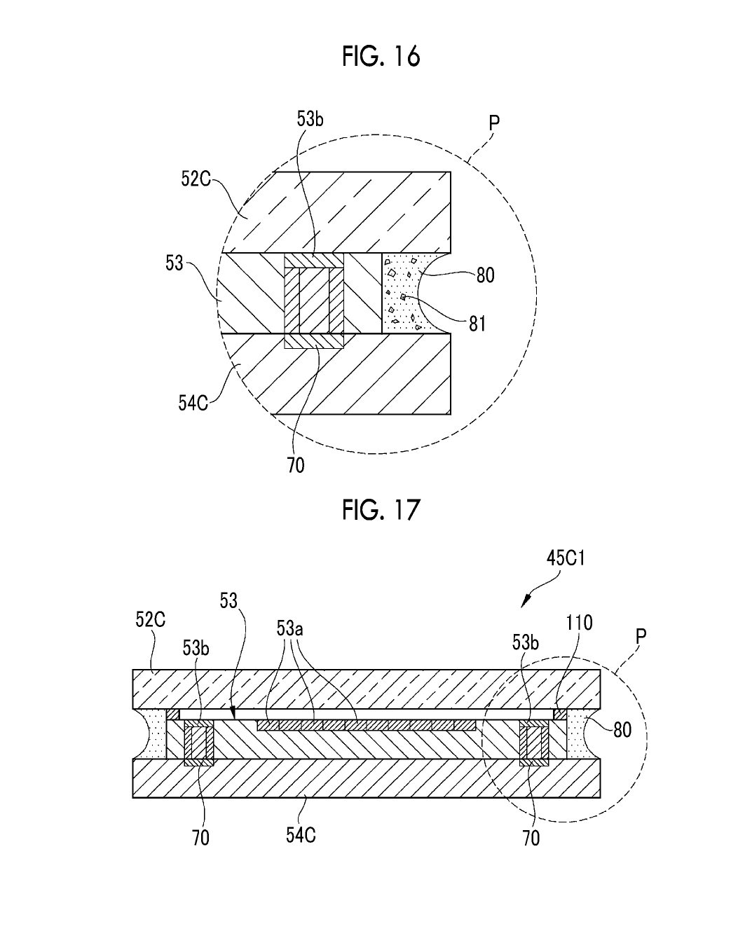

FIG. 15 is an enlarged view of the main part (the cover glass 52C, the imaging device 53, and the back substrate 54C) of the imaging apparatus 45C in FIG. 14. FIG. 16 is an enlarged view of a portion surrounded by a dotted circle P in the imaging apparatus 45C shown in FIG. 15. As shown in FIGS. 15 and 16, a gap is formed between the cover glass 52C and the back substrate 54C by the cover glass 52C, the back substrate 54C, and the side surface of the imaging device 53, and a sealing resin 80 containing a filler 81 fills the gap using a capillary phenomenon based on surface tension and is solidified.

In this case, in the imaging apparatus 45C, since the sizes of the outer shapes of the cover glass 52C and the back substrate 54C are the same, the sealing resin 80 is provided on the inner side rather than the outer peripheries of the cover glass 52C and the back substrate 54C.

Thus, also in the imaging apparatus 45C of the fourth embodiment, the outer shapes of the cover glass 52C and the back substrate 54C are formed so as to be larger than the outer shape of the imaging device 53, and the side surface of the imaging device 53 is sealed by filling a gap between the cover glass 52C and the back substrate 54C with the sealing resin 80. Therefore, it is possible to obtain the same effects as those described in the first embodiment.

[Modification Example of the Imaging Apparatus of the Fourth Embodiment]

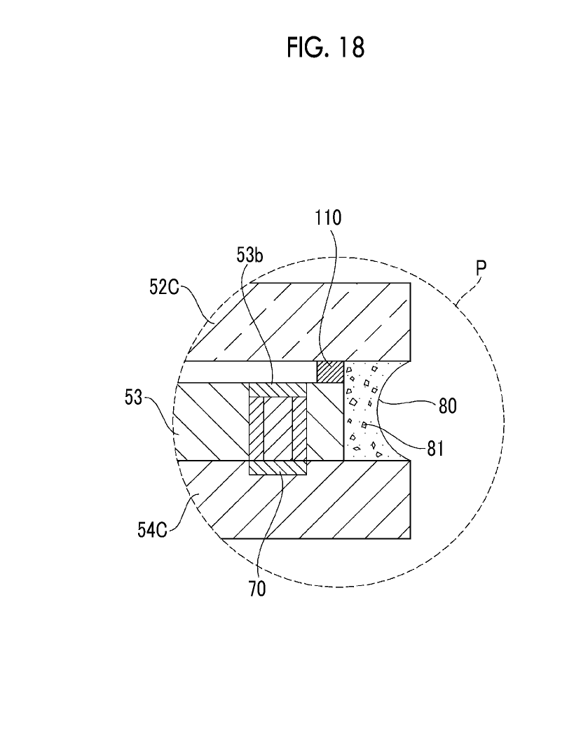

Next, an imaging apparatus 45C1 that is a modification example of the imaging apparatus 45C of the fourth embodiment of the disclosure will be described with reference to FIGS. 17 and 18. FIG. 17 is a cross-sectional view of the imaging apparatus 45C1, and FIG. 18 is an enlarged view of a portion surrounded by a dotted circle P in the imaging apparatus 45C1 shown in FIG. 17. Although the cover glass 52C is directly fixed to the imaging surface of the imaging device 53 in the imaging apparatus 45C of the fourth embodiment described above, a spacer 110 is provided between the cover glass 52C and the imaging device 53 in the imaging apparatus 45C1.

Since the imaging apparatus 45C1 has basically the same configuration as the imaging apparatus 45C of the fourth embodiment except that the spacer 110 is provided, components having the same functions and configurations as in the fourth embodiment described above are denoted by the same reference numerals, and explanation thereof will be omitted.

The sealing resin 80 is located between the cover glass 52C and the back substrate 54C, and is solidified after filling a gap formed by the cover glass 52C, the back substrate 54C, the side surface of the imaging device 53, and the spacer 110. Therefore, since the side surface of the imaging device 53 is sealed, it is possible to obtain the same effects as those described in the first embodiment.

Also in the imaging apparatuses of the first to third embodiments, it is possible to provide a spacer between the cover glass and the imaging device as in the imaging apparatus 45C1.

[Example of Application to a Capsule System of an Imaging Apparatus]

As electronic endoscopes in which the imaging apparatuses configured as described above are mounted, a soft endoscope, a hard endoscope, an industrial endoscope, a capsule system (also referred to as a capsule type endoscope), and the like can be used. Hereinafter, a capsule system will be described in detail as an example with reference to the accompanying diagrams.

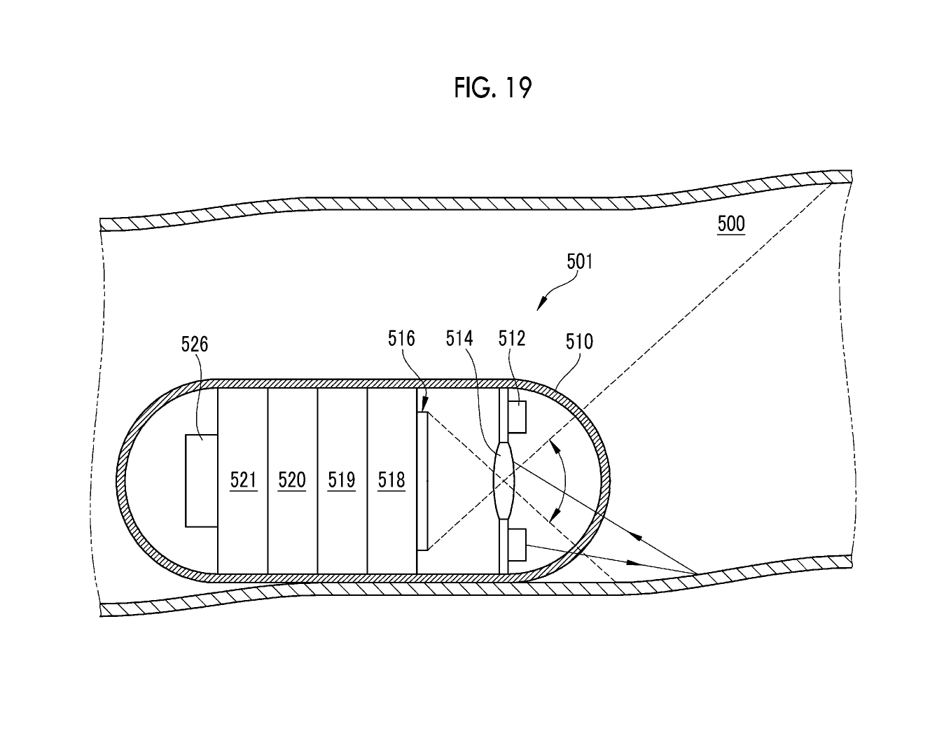

As shown in FIG. 19, a capsule system 501 includes an illumination system 512 and a camera including an optical system 514 and an image sensor 516. An image captured by the image sensor 516 is processed by an image processor 518. The image processor 518 can be implemented by software executed by a digital signal processor (DSP) or a central processing unit (CPU), or by hardware, or by a combination of both software and hardware. The processed image is compressed by an image compression sub-system 519 (may be mounted in software executed by the DSP of the image processor 518 depending on an embodiment). The compressed data is stored in an archive memory system 520. The capsule system 501 includes a battery power supply 521 and an output port 526. The capsule system 501 can move through the gastrointestinal tract (GI tract) 500 by peristalsis.

As the illumination system 512, an LED can be mounted. In FIG. 19, the LED is disposed close to the opening of a camera. However, other arrangements can also be adopted. For example, a light source may be provided behind the opening. Other light sources, such as a laser diode, may also be used. Alternatively, a combination of two or more narrow wavelength band light sources or a white light source can also be used. In order to emit light having a long wavelength, it is possible to use a white LED together with a phosphorescent material that is excited by light of the LED. The white LED may include a blue LED or a purple LED. A predetermined portion of a capsule housing for passing the light is formed of glass or polymer that is biologically suitable.

The optical system 514 is for reading an image of a wall of the lumen, such as the GI tract 500, into the image sensor 516, and may include a plurality of refractive lens elements, diffractive lens elements, or reflective lens elements.

The image sensor 516 converts the received light into a corresponding electrical signal, and can be provided by a charge coupled device (CCD) or complementary metal oxide semiconductor (CMOS) type device. The image sensor 516 may be a sensor that responds to a single color, or may include a color filter array that can capture a color image (for example, using RGB or CYM expression).

The image sensor 516 is equivalent to one form of an imaging apparatus of the disclosure, and is configured to include basically the same cover glass, imaging device, and back substrate as in the imaging apparatus of each of the embodiments described above. Also in the image sensor 516, the outer shapes of the cover glass and the back substrate are formed so as to be larger than the outer shape of the imaging device, and the side surface of the imaging device is sealed by filling a gap between the cover glass and the back substrate with the sealing resin. Therefore, it is possible to obtain the same effects as those described in each of the embodiments.

The analog signal from the image sensor 516 is preferably converted into a digital form so as to be able to be processed in a digital format. Such conversion is performed using an analog-to-digital (A/D) converter provided in a sensor (in the case of the present embodiment) or in another portion of a capsule housing 510. The A/D unit may be provided between the image sensor 516 and another portion of the system. The LED of the illumination system 512 is synchronized with the operation of the image sensor 516. One of the functions of a control module (not shown) of the capsule system 501 is to control the LED during the operation of capturing an image.

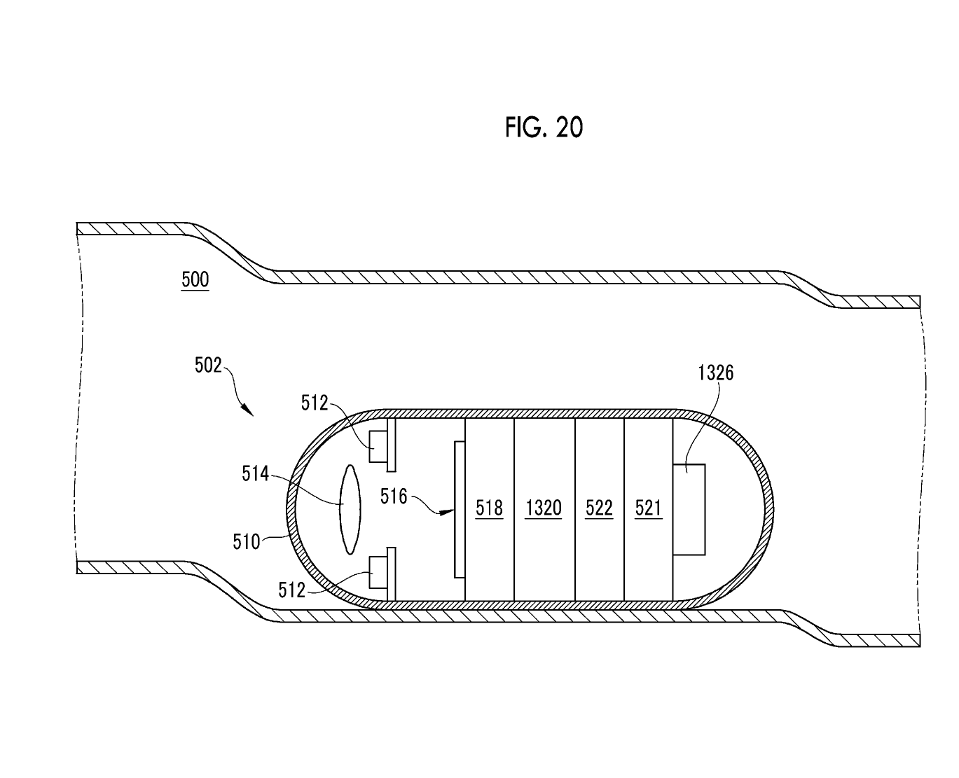

FIG. 20 shows a swallowable type capsule system 502 according to an embodiment of the disclosure. The capsule system 502 can be made to have substantially the same configuration as the capsule system 501 shown in FIG. 19 except that the archive memory system 520 and the output port 526 are not required. The capsule system 502 also includes a communication protocol encoder 1320 and a transmitter 1326 that are used for wireless transmission. Among the elements of the capsule system 501 and the capsule system 502, substantially the same elements are denoted by the same reference numerals. Accordingly, their structures and functions will not be described again herein. A control module 522 performs overall control of the capsule system 502. The communication protocol encoder 1320 is implemented by software executed by the DSP or the CPU, or by hardware, or by a combination of both software and hardware. The transmitter 1326 includes an antenna system for transmitting a captured digital image.

[Others]

Although the electronic endoscope including the imaging apparatus of the disclosure has been described in each of the embodiments, the imaging apparatus of the disclosure can be provided in various apparatuses and systems, such as a digital camera including an imaging device.

It is needless to say that the disclosure is not limited to the embodiments described above and various modifications can be made within the scope and spirit of the disclosure.

What is claimed is:

1. An imaging apparatus, comprising:
an imaging device in which a plurality of photoelectric conversion elements are arrayed;
a substrate on which the imaging device is provided and which has a larger outer shape than the imaging device;
a transparent cover member that is provided on an opposite surface side to a surface of the imaging device facing the substrate and has a larger outer shape than the imaging device; and
a sealing resin that fills a gap between the substrate and the transparent cover member in order to seal a side surface of the imaging device.

2. The imaging apparatus according to claim 1, wherein
the sealing resin is provided on an inner side rather than outer peripheries of the substrate and the transparent cover member, when sizes of the outer shapes of the substrate and the transparent cover member are the same.

3. The imaging apparatus according to claim 1, wherein
when sizes of the outer shapes of the substrate and the transparent cover member are different, the sealing resin is provided on an inner side rather than an outer periphery of one of the substrate and the transparent cover member having the larger outer shape.

4. The imaging apparatus according to claim 3, wherein
a pattern that has a frame shape surrounding the sealing resin and regulates a flow of the sealing resin toward the outer periphery of one of the substrate and the transparent cover member having the larger outer shape is formed on a surface, which faces the imaging device, of one of the substrate and the transparent cover member having the larger outer shape.

5. The imaging apparatus according to claim 4, wherein
the pattern is formed by plating or deposition when the transparent cover member has a larger outer shape.

6. The imaging apparatus according to claim 1, wherein
the sealing resin contains a filler having higher thermal conductivity than the sealing resin.

7. The imaging apparatus according to claim 2, wherein
the sealing resin contains a filler having higher thermal conductivity than the sealing resin.

8. The imaging apparatus according to claim 3, wherein
the sealing resin contains a filler having higher thermal conductivity than the sealing resin.

9. The imaging apparatus according to claim 4, wherein
the sealing resin contains a filler having higher thermal conductivity than the sealing resin.

10. The imaging apparatus according to claim 5, wherein
the sealing resin contains a filler having higher thermal conductivity than the sealing resin.

11. The imaging apparatus according to claim 1, wherein
a tensile modulus of elasticity of the sealing resin is 100 Mpa or less.

12. The imaging apparatus according to claim 2, wherein
a tensile modulus of elasticity of the sealing resin is 100 Mpa or less.

13. The imaging apparatus according to claim 3, wherein
a tensile modulus of elasticity of the sealing resin is 100 Mpa or less.

14. The imaging apparatus according to claim 4, wherein
a tensile modulus of elasticity of the sealing resin is 100 Mpa or less.

15. The imaging apparatus according to claim 5, wherein
a tensile modulus of elasticity of the sealing resin is 100 Mpa or less.

16. The imaging apparatus according to claim 6, wherein
a tensile modulus of elasticity of the sealing resin is 100 Mpa or less.

17. The imaging apparatus according to claim 1, wherein
a through wiring portion is formed in the imaging device, and a connecting portion electrically connected to the through wiring portion is formed on a surface of the substrate facing the imaging device, and
the imaging device and the substrate are electrically connected to each other through the through wiring portion and the connecting portion.

18. The imaging apparatus according to claim 1, wherein
the imaging device is of a Back Side Illumination type.

19. The imaging apparatus according to claim 1, wherein
a spacer is provided between the imaging device and the transparent cover member.

20. The imaging apparatus according to claim 1, wherein
the transparent cover member is formed of glass or transparent resin.
